(19) United States
(12) Patent Application Publication
(10) Pub. No.: US 2008/0064608 A1
(43) Pub. Date: Mar. 13, 2008

(54) Method Of Detecting Phosphorylation By Spr Using Zinc Chelating Reagent

(75) Inventors: Kazuki Inamori, Fukui (JP); Yoshiaki Nishiya, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: TOYO BOSEKI KABUSHIKI KAISHA, Osaka (JP)

(21) Appl. No.: 11/631,978

(22) PCT Filed: Jul. 6, 2005
(86) PCT No.: PCT/JP05/12451

§ 371 Date: Jan. 9, 2007

(30) Foreign Application Priority Data
Jul. 13, 2004 (JP) 2004-205562
Aug. 11, 2004 (JP) 2004-234635
Nov. 8, 2004 (JP) 2004-323530
Nov. 8, 2004 (JP) 2004-323531

(51) Int. Cl. C40B 30/04; C12Q 1/48; G01N 33/00

(52) U.S. Cl. 506/9; 435/15; 435/7.1

(57) ABSTRACT

[Problems] To provide a method which comprises detecting phosphorylation of substrate peptides with a quick and easy scheme using an inexpensive substance without a need for a special technique and which among other things enables comprehensive profiling kinetics of various protein kinases.
[Means for Solving Problems] A method for analysis of protein kinase activity by detecting phosphorylation of at least one peptide which serves as a substrate for at least one protein kinase and is immobilized on the metal thin film in a basal plate of an array, characterized in that, in detecting the phosphorylation, the phosphorylated peptide is treated with a polyamine-zinc complex represented by, e.g., formula (I) which has a molecular weight of 500 to 1,000 and is modified with biotin and then preferably with avidin or streptavidin.

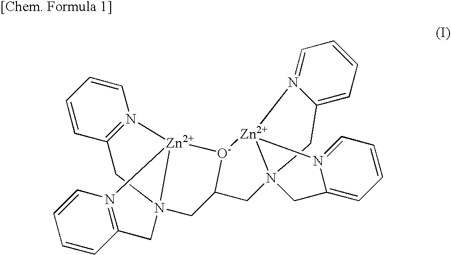

### TECHNICAL FIELD

The present invention relates to a method of analysis of protein kinase activity using an array containing a metal basal plate on which one or more peptides which is recognized by a protein kinase are immobilized. The method detects phosphorylation by phosphorylation on an array and treatment with a chelate compound modified with biotin. In particular, the invention relates to a quick and efficient method which realizes simple analysis of protein kinase activity with analysis technology using surface plasmon resonance (hereinafter, may be referred to as “SPR”). More specifically, the method is a novel analysis technique for protein kinase activity and an application of surface plasmon resonance imaging (hereinafter, may be referred to as “SPR imaging”) which produces images based on SPR analysis of interaction between substances.

### BACKGROUND ART

Recent years have seen dramatic progress in the studies of intercellular signaling. We now have a better understanding about how a signal is transduced from a receptor on a surface of a cell activated by a growth factor, cytokine, etc. to the nucleus. Apart from that, we have increasingly precise knowledge on various signaling pathways which control, for example, cell cycle, adhesion, motion, polarity, morphogenesis, differentiation, and life and death. These signaling pathways do not function individually, but undergo crosstalk, thereby functioning as a system. Cancer and many other disease are attributed to abnormality of the signaling pathways.

It is known that various protein kinases act together in a complicated manner to play important roles in the signaling pathways. It is expected that it will be a great contribution to drug development, clinical application, and other related fields, as well as to basic study in cell biology and pharmacology if the activity of these protein kinases is comprehensively analyzed and their intercellular kinetics are profiled all at once. So far, however, there has been no simple and efficient technique established for simultaneous profiling of kinetics of various protein kinases.

A related technique is reported in, for example, non-patent document 1. The technique uses, for example, a peptide array to evaluate the activity of cSrc kinase, which is a tyrosine kinase. Also, a detection system for phosphorylation reaction using a fluorescence-labeled antibody is reported in, for example, non-patent documents 2 and 3. In the system, arrays are used which contain a glass slide on which substrate peptides for p60 tyrosine kinase, protein kinase A (hereinafter, may be referred to as “PKA”), etc. are immobilized. Further, a detection system for kinase reaction on an array using a radioactive substance ([γ³²P]ATP) is reported in, for example, non-patent documents 4, 5, and 6. None of these prior art documents discloses a sufficient technique for simultaneous and efficient profiling of kinetics of various protein kinases. Moreover, the methods have serious issues. For example, the methods require the use of fluorescent or radioactive substance, which makes the analysis laborious, adds difficulty to handling, and calls for the use of special techniques and equipment.

The detection system using an antibody is applied in various ways. However, no antibody has been discovered which in particular recognizes phosphorylated serine or phosphorylated threonine sufficiently in terms of binding specificity and affinity. The detection system therefore has large problems before we can achieve high precision measurement with it. The antibody is not very advantageous to use as a universal detection system means because different antibodies need to be used to treat different phosphorylated amino acids, and their binding is often highly dependent on the amino acid sequence in the neighborhood of a phosphorylated amino acid.

[Non-patent Document 1] Benjamin T. Houseman, et al., Nature Biotechnology, Vol. 20, pp. 270-274 (March 2002)

[Non-patent Document 2] Bioorganic & Medical Chemistry Letters, Vol. 12, pp. 2,085-2,088 (2002)

[Non-patent Document 3] Bioorganic & Medical Chemistry Letters, Vol. 12, pp. 2,079-2,083 (2002)

[Non-patent Document 4] Current Opinion in Biotechnology, Vol. 13, pp. 315-320 (2002)

[Non-patent Document 5] Journal of Biological Chemistry, Vol. 277, pp. 27,839-27,849 (2002)

[Non-patent Document 6] Science, Vol. 289, pp. 1,760-1,763 (2000)

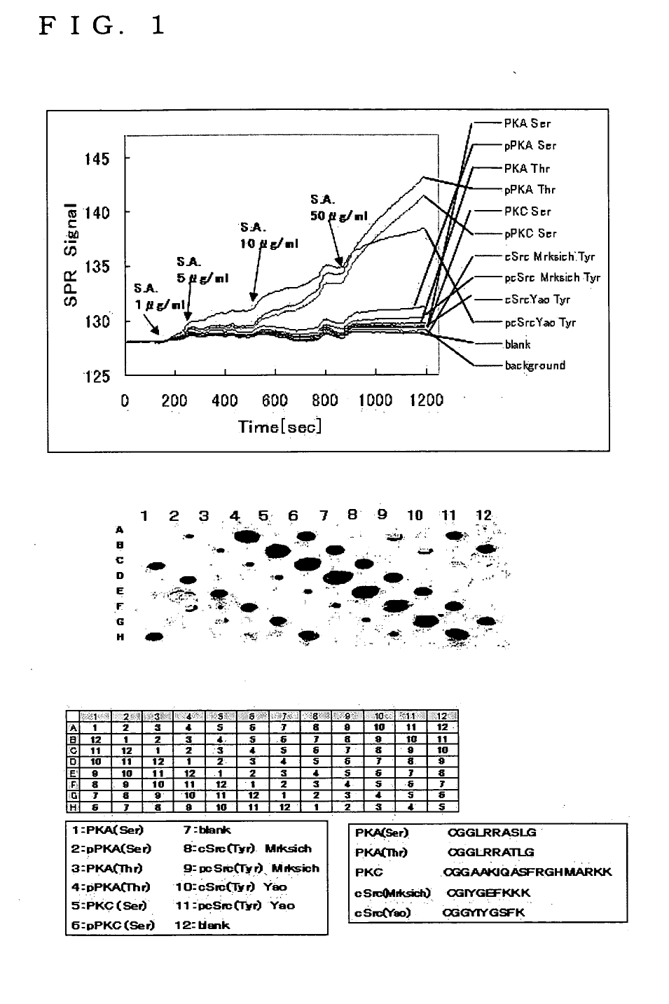

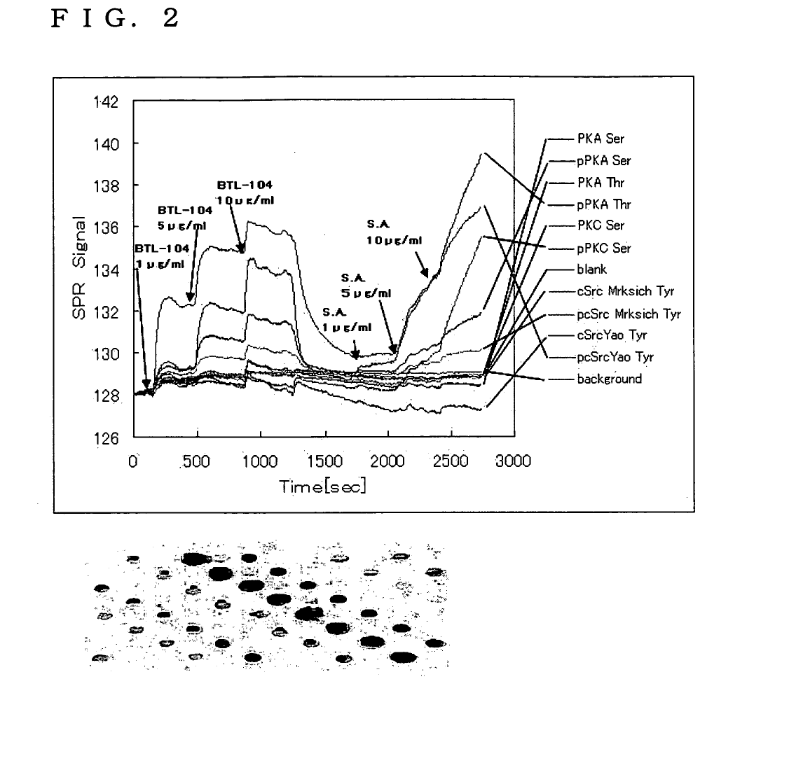

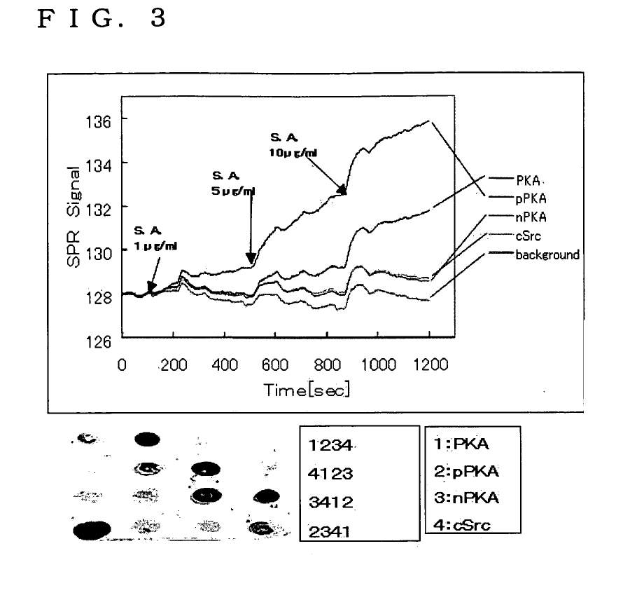

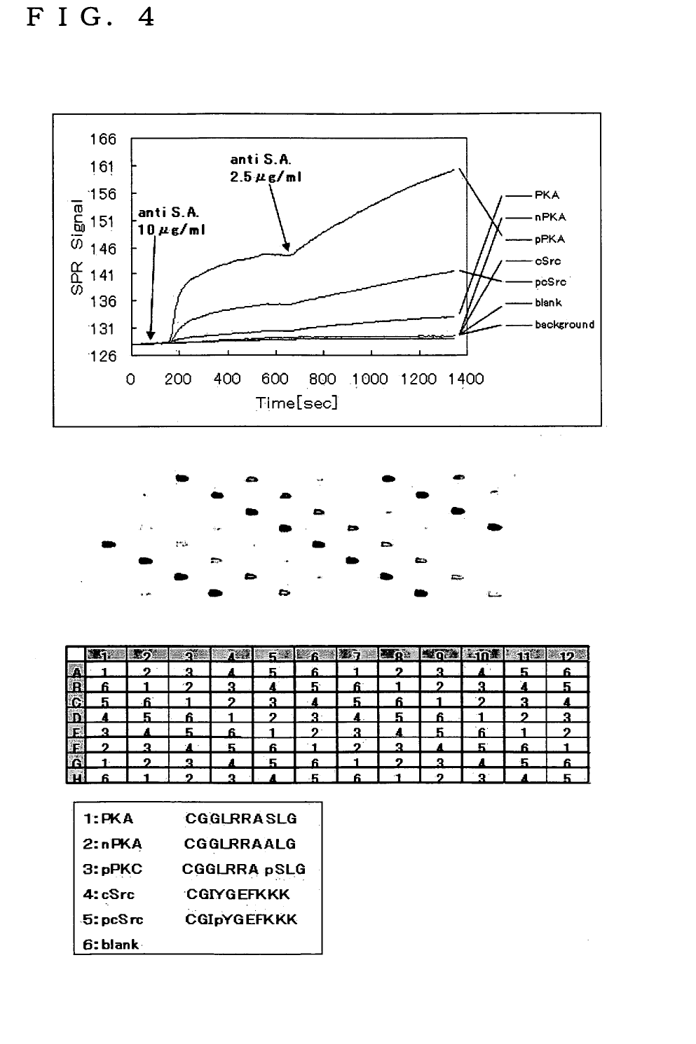

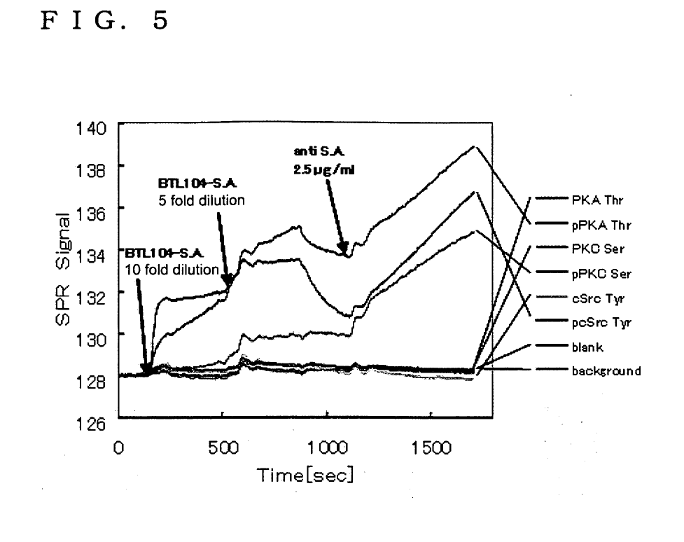

### BRIEF DESCRIPTION OF DRAWINGS

[FIG. 1] A drawing, for example 1, showing array patterns of immobilized substrate peptides and results of SPR analysis and SPR imaging.

[FIG. 2] A drawing, for example 2, showing results of SPR analysis and SPR imaging.

[FIG. 3] A drawing, for example 3, showing array patterns of immobilized substrate peptides and results of SPR analysis and SPR imaging.

[FIG. 4] A drawing, for example 4, showing array patterns of immobilized substrate peptides and results of SPR analysis and SPR imaging.

[FIG. 5] A drawing, for example 5, showing results of SPR analysis.

### DISCLOSURE OF THE INVENTION

Problems to Be Solved by the Invention

The present invention is intended to establish a method of comprehensive profiling of, especially, kinetics of various protein kinases by detecting phosphorylation of a substrate peptide with a quick and easy scheme using an inexpensive substance without a need for a special technique.

Means to Solve Problems

The inventors, in view of the circumstances outlined above, have diligently worked and as a result found that for quick and, especially, comprehensive analysis of kinetics of various protein kinases, it is extremely useful to phosphorylate with a protein kinase using an array containing a basal plate carrying thereon a vapor-deposited metal on which is immobilized a peptide which is recognized by the protein kinase and thereafter detect interaction between substances, especially, on the array with SPR imaging upon treating with a biotin-modified chelate compound having a particular molecular weight, and preferably further treating with streptavidin or avidin, which has led to the completion of the invention.

The present invention has the following features:

1. A method of analysis of protein kinase activity, being characterized by, in judging phosphorylation using a peptide which is immobilized on a basal plate, contacting a chelate compound modified with a ligand with the object peptide on the basal plate.

2. The method of entry 1, wherein after the treating with the chelate compound modified with a ligand, the peptide is further treated with a receptor.

3. The method of either one of entries 1 and 2, wherein after the treating with the receptor, the peptide is further treated with an antibody which recognizes the receptor.

4. A method of analysis of protein kinase activity, being characterized by, in judging phosphorylation using a peptide which is immobilized on a basal plate, forming a complex of a receptor and a chelate compound modified with a ligand and contacting the complex with the object peptide on the basal plate.

5. The method of entry 4, wherein after the treating with the complex, the peptide is further treated with an antibody which recognizes the receptor.

6. The method of any one of entries 1 to 5, wherein the chelate compound modified with a ligand has a molecular weight of 500 to 1,000.

7. The method of entry 6, wherein the chelate compound modified with a ligand has a molecular weight of 600 to 900.

8. The method of any one of entries 1 to 7, wherein: the ligand is biotin; and the receptor specific to that ligand is either avidin or streptavidin.

9. The method of any one of entries 1 to 8, wherein the chelate compound is a polyamine-zinc complex.

10. The method of any one of entries 1 to 9, wherein the chelate compound is a binuclear zinc complex containing a polyamine compound as a chelator.

11. The method of any one of entries 1 to 10, wherein the chelate compound is a compound of formula (I): 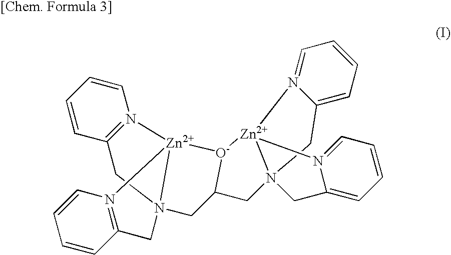
12. The method of any one of entries 1 to 11, wherein the peptide is a substrate for at least one protein kinase selected from the group consisting of the cGMP-dependent protein kinase family, the cAMP-dependent protein kinase (PKA) family, the myosin light chain kinase family, the protein kinase C (PKC) family, the protein kinase D (PKD) family, the protein kinase B (PKB) family, the protein kinase family belonging to the MAP kinase (MAPK) cascade, the Src tyrosine kinase family, and the receptor tyrosine kinase family.
13. The method of any one of entries 1 to 12, wherein an array is used which contains at least two peptides immobilized on a metal thin film, each peptide being a substrate for a different protein kinase.
14. The method of any one of entries 1 to 13, wherein phosphorylation of at least one peptide which serves as a substrate for at least one protein kinase and is immobilized on a metal thin film in a basal plate of an array is detected by treating the at least one peptide with a nucleoside triphosphate and a test material which can contain a protein kinase.
15. The method of any one of entries 1 to 14, wherein a phosphorylated peptide is detected by surface plasmon resonance (SPR).
16. The method of any one of entries 1 to 15, wherein a phosphorylated peptide is detected by surface plasmon resonance imaging.
17. The method of either one of entries 1 and 4, wherein the basal plate has a metal thin film, and the peptide is immobilized on the metal thin film.
18. The method of any one of entries 1, 4, and 17, wherein the peptide is immobilized on the basal plate, forming an array.
19. The method of entry 18, wherein two or more peptides are immobilized, forming an array.
20. The method of entry 18, wherein two or more peptides are immobilized, forming an array, the two or more peptides each containing an amino acid residue which is a combination of any two or more of serine, threonine, and tyrosine, the residue providing a site where the peptide is phosphorylated.
21. A kit for detecting phosphorylation using a peptide which is immobilized on a basal plate, the kit comprising: a chelate compound modified with a ligand; and a receptor specific to the ligand.
22. The kit of entry 21, the kit further comprising an antibody which recognizes the ligand.
23. The kit of either one of entries 21 and 22, wherein: the ligand is biotin; and the receptor specific to that ligand is either avidin or streptavidin.
24. The kit of any one of entries 21 to 23, wherein the chelate compound is a polyamine-zinc complex.
25. A kit for detecting phosphorylation using a peptide which is immobilized on a basal plate, the kit comprising a complex of a receptor and a chelate compound modified with a ligand.
26. The kit of entry 25, the kit further comprising an antibody which recognizes the ligand.
27. The kit of either one of entries 25 and 26, wherein: the ligand is biotin; and the receptor specific to that ligand is either avidin or streptavidin.
28. The kit of any one of entries 25 to 27, wherein the chelate compound is a polyamine-zinc complex.
29. The kit of any one of entries 21 to 28, wherein the chelate compound is a binuclear zinc complex containing a polyamine compound as a chelator.
30. The kit of any one of entries 21 to 29, wherein the chelate compound is a compound of formula (I): 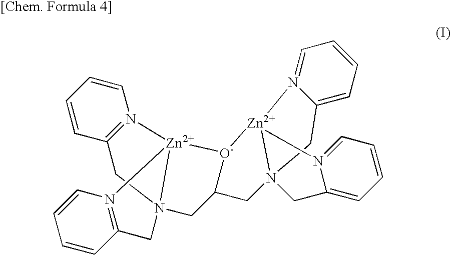
31. The kit of any one of entries 21 to 30, wherein a phosphorylated form of the peptide is detected by surface plasmon resonance (SPR).
32. The kit of any one of entries 21 to 31, wherein a phosphorylated form of the peptide is detected by SPR imaging.
Effects of the Invention

The method of the present invention enables very simple and quick analysis of kinetics of various protein kinases without a need for a special technique. If SPR is used together with the method, there is no need to use a label either, such as a fluorescent or radioactive substance. The use of a chelate compound reduces cost and enables easy handling. Moreover, owing to that use, the method is not affected by the type of phosphorylated amino acid or the amino acid sequence in its neighborhood. These are great advantages over conventional methods. Especially, the present invention enables comprehensive analysis of many types of protein kinase signals, which leads to effective profiling of intercellular protein kinase kinetics upon drug administration or upon the introduction of a gene of which the functions are unknown. Accordingly, the invention is expected to find applications in genome drug development, for example, to analyze functions of new genes and as drug research tools.

Best Mode for Carrying Out the Invention

The method of detecting a phosphorylated form of a peptide on a basal plate in accordance with the present invention may involve the use of a conventionally well-known label compound, such as a radioactive substance, a fluorescent substance, or a chemiluminescent substance. Preferably, however, an optical detection method is used: e.g., surface plasmon resonance (SPR), ellipsometry, or sum frequency generation (hereinafter, “SFG”) spectrometry. Especially preferred among them is SPR because there is no need to obtain phase difference. SPR is capable of detection of changes in surface film thickness on the order of nanometers by obtaining the intensity of reflected light alone. SPR imaging is preferable because the method allows observation of a large area and also observation of substance interaction using an array.

A peptide can be phosphorylated by applying a nucleoside triphosphate, such as ATP, and a test sample which can contain a protein kinase onto the array of the present invention. Optimal conditions for phosphorylation reaction vary with the type of protein kinase. As an example, the peptide is phosphorylated by adding a nucleoside triphosphate and a test sample which can contain a protein kinase into a buffer and letting them react at about 10 to 40° C., preferably 30 to 40° C., for about 10 minutes to 6 hours, preferably for about 30 to 1 hour. A phosphorylation-facilitating substance, such as cAMP, cGMP, Mg²⁺, or Ca²⁺, phospholipid, may be added, where necessary, to the reaction solution for phosphorylation.

The protein kinases that are targets in kinetics profiling are, for example, the enzymes that phosphorylate a side chain of amino acids, such as tyrosine, serine, threonine, and histidine in protein. More specific examples are the cGMP-dependent protein kinase family, the cAMP-dependent protein kinase (PKA) family, the myosin light chain kinase family, the protein kinase C (PKC) family, the protein kinase D (PKD) family, the protein kinase B (PKB) family, the protein kinase family belonging to the MAP kinase (MAPK) cascade, the Src tyrosine kinase family, and the receptor tyrosine kinase family.

The peptide in the present invention is aimed at making comprehensive profiling of kinetics of the protein kinases. So, each peptide is preferably phosphorylated by only one protein kinase, and not by other protein kinases. The peptide sequence used as a substrate for the protein kinase may have a publicly known sequence or a suitable one selected from variations of the publicly known sequence. The array of the present invention preferably carries immobilized peptides corresponding to the plurality of protein kinases of which the kinetics need to be understood because the use of only one such array enables the profiling of all the protein kinases. Of course, it is possible to immobilize on each array a peptide corresponding to only one protein kinase and use a required number of arrays when profiling the protein kinases.

Ellipsometry measures the thickness and refractive index of a thin film through changes in polarization caused by interference between reflections of light irradiated onto a sample, one from the front surface of the thin film and the other from the back surface of the thin film. Ellipsometry provides a means of evaluating the ratio of the absolute value of reflectance for primary polarized light and that for secondary polarized light and the ratio of phase shifts for the primary and secondary polarized light. Spectroscopic ellipsometry, or ellipsometry with variable wavelengths, is especially preferred because the method detects changes in the surface film thickness with high sensitivity.

SFG is a secondary non-linear optical effect. The term refers to a phenomenon of two incident beams of light at different frequencies, ω1 and ω2, producing light at ω1+ω2 or ω1−ω2 when mixed in a medium. If visual light and wavelength-variable infrared light are used for ω1 and ω2 respectively, it is possible to carry out vibrational spectroscopy, which resembles infrared spectroscopy. This technique, with its high surface selectivity, enables vibrational spectroscopy on molecules in a single-molecule-thick layer. It is a useful surface analysis method with high sensitivity.

As mentioned above, in one preferred embodiment, the present invention is especially preferably adapted to comprehensively analyze the activity of various protein kinases by SPR. In SPR, a flux of polarized light is irradiated onto metal, producing evanescent waves. The waves then reach to the surface and excite surface plasmons (surface waves). The plasmons consume the light's energy and reduces the intensity of reflection. The resonance angle, or the angle at which the reflection intensity drops by a large amount, changes with the thickness of the layer formed on the metal surface. By observing changes in the resonance angle or changes in the reflection intensity at an angle, one can detect interaction between a target substance (or a collection of substances) immobilized on the metal surface and another substance (or a collection of substances) in a sample. Therefore, SPR requires no labeling with, for example, a fluorescence or radioactive substance. It is a useful assay system capable of real time evaluation.

SPR imaging, an application of SPR, irradiates a flux of polarized light over a large area to analyze images of its reflection. In doing so, the method makes full use of image processing and other related techniques to produce images showing the interaction between the substances. The method enables screening on a chip carrying more than one substance immobilized on it and also high sensitivity observation of the morphology of objects adsorbing to the surface.

In SPR imaging, a means is needed that irradiates the flux of polarized light with sufficient intensity over a large area of the chip for the analysis of reflection images. FIG. 1 shows an example of such a means. The intensity of the polarized light flux is preferably higher because it delivers higher sensor sensitivity.

The light source may be of any type, not limited in any particular manner. A preferable light source however includes near-infrared wavelengths at which the changes in the SPR resonance angle are especially sensitive. Specific examples include a metal halide lamp, a mercury lamp, a xenon lamp, a halogen lamp, a fluorescent lamp, an incandescent lamp, or another white light emitting source that irradiates over a large area. Especially preferred among them is the halogen lamp, which produces light with the required high intensity and operates from a simple, inexpensive power supply.

The filament of a common white light emitting source has such a flaw that it emits light with non-uniform brightness. If the light emitted from the light source is irradiated as it is, the reflection image is non-uniform in brightness. That would make it difficult to perform screening and evaluate morphology changes. Therefore, the emitted light is preferably guided through a pinhole before being collimated, so that the chip can be illuminated with uniform light. Guiding light through a pinhole is a preferable means of obtaining a flux of light with uniform brightness. The means however undesirably reduces the light's intensity if the light is guided unaided through the pinhole. To address the problem and secure sufficient intensity, a convex lens is preferably disposed between the light source and the pinhole to collect the light before guiding the light through the pinhole.

Since the emitted white light is non-directional, it needs to be collimated through another convex lens before being collected. The light source is disposed about the focal length away from the convex lens to produce parallel light. The first convex lens is disposed with the pinhole being positioned about the focal length away from the lens, so that the light can be collected at the pinhole. The light crosses as it passes inside the pinhole. After that, the light is collimated through a CCTV camera lens. The cross-sectional area of the obtained parallel light is preferably adjusted to 10 to 1,000 mm². This way, it becomes possible to perform screening and morphology observation over a large area.

In imaging the interaction, the flux of polarized light is irradiated to a side of the metal thin film opposite to the side on which the substance or the collection of substances are immobilized. The flux is reflected off the film and guided through an optical interference filter to obtain near-infrared light. The remaining light, in a certain limited region of the spectrum, is captured on a CCD camera.

The optical interference filter preferably has a central wavelength of 600 to 1,000 nm at which SPR shows high sensitivity. The transmittance of the optical interference filter falls to half the maximum value at wavelengths termed half width. The smaller the half width, the sharper the distribution with respect to wavelength. A smaller half width is therefore preferable. Specifically, the half width is preferably 100 nm or less. The image filtered by the optical interference filter and captured on the CCD camera is fed to a computer on which one can evaluate brightness changes in a part of the image on real time or across the image by image processing. Thus, it becomes possible to perform screening on a chip carrying more than one substance immobilized on it, as well as high sensitivity observation of the morphology of objects adsorbing to the surface.

The SPR chip, or slide, used in the present invention preferably consists of a metal basal plate which has a metal thin film provided on a transparent basal plate. A substance or a collection of substances are chemically or physically immobilized directly or indirectly onto the metal thin film. The basal plate may be made of any material; it is preferably made of a transparent material. Specific examples are glass and plastics, such as polyethylene terephthalate (PET), polycarbonate, and acrylic. Glass is especially preferred among them.

The basal plate is preferably about 0.1 to 20 mm thick, more preferably 1 to 2 mm thick. For the purpose of evaluating the reflection image of the metal thin film, the SPR resonance angle should be as small as possible so that the captured image does not deform. Easy analysis is thus achieved. Therefore, the transparent basal plate, or the transparent basal plate and the prism in contact with that basal plate, preferably has/have a refractive index, nD, of 1.5 or higher.

The metal thin film is made of, for example, gold, silver, copper, aluminum, platinum, or any combination of these metals. Gold is especially preferred. The metal thin film may be fabricated in any manner. Examples of publicly known methods include vapor deposition, sputtering, and ion coating. Vapor deposition is a preferred method. The metal thin film is preferably about 10 to 3,000 Å thick, more preferably about 100 to 600 Å thick.

A preferred concrete example of the present invention is characterized as follows. The examples uses an array containing a basal plate carrying thereon a vapor-deposited metal on which is immobilized at least one peptide (preferably two or more peptides) which serves as a substrate for a protein kinase. The array is treated with a solution containing a kinase, such as a homogenized cell solution. The array is then treated with a chelate compound. Their interaction is detected by SPR or SPR imaging among other techniques. In the present invention, the peptide which serves as a substrate for a protein kinase refers to a peptide which is phosphorylated by the protein kinase.

The peptide is not limited in terms of length in any particular manner. The peptide typically contains 100 or less, preferably about 5 to 60, more preferably about 10 to 25 amino acid residues. The peptide may be obtained by chemical synthesis using publicly known techniques or fabricated by genetic engineering techniques. To help the peptide to bind to, or part with, a basal plate, the peptide may have biotin, cysteine residue with a thiol group, or a common tag, such as oligohistidine (His-tag) or glutathione-S-transferase (GST), added one of the termini.

The substrate peptide may be immobilized on the metal thin film by any method. Preferably, however, a functional group which is readily immobilized on the surface of the metal thin film is introduced prior to the immobilization of the peptide. Examples of such a functional group include an amino group, a mercapto group, a carboxyl group, and an aldehyde group. These functional groups are preferably introduced to the surface of the metal thin film by using a common alkanethiol derivative.

The use of the alkanethiol derivative may be based on the method described by J. M. Brockman, et al., J. Am. Chem. Soc. Vol. 121, pp. 8,044 to 8,051 (1999), so as to immobilize the peptide to the surface with an intervening alkanethiol layer between them and then modify the background with PEG (polyethylene glycol). Also, the peptide may be immobilized after being bound, to the alkanethiol, a derivative in which a functional group listed above is introduced to the end of PEG. This is useful to prevent nonspecific effects and for spacer effect.

Specifically, for example, a water-soluble polymer, like PEG, which has its terminus being modified with carboxymethyl dextran or a carboxyl group is immobilized to the metal thin film to introduce a carboxyl group to the surface. Then, using water-soluble carbodiimide, like EDC (1-ethyl-3,4-dimethylaminopropyl carbodiimide), an amino group in the peptide or protein can react with the activated carboxyl group such as NHS (N-hydroxysuccinimide) ester. Alternatively, the surface is modified with maleimide, and the peptide is immobilized on the surface with intervening amino acid residues containing cysteine or another thiol group. The cysteine residue in that case is preferably added to one of the termini of the peptide. Between these immobilization methods, the latter, which involves an intervening thiol group, is preferred for reduction in nonspecific reactions. These examples are however not limiting the invention in any particular manner.

The aforementioned method of immobilizing a peptide tagged with His-tag or GST is also very simple and useful. In that case, an amino group or a carboxyl group is preferably introduced to the metal surface with intervening alkanethiol as mentioned above, after which NTA (nitrilotriacetic acid) or glutathione is introduced respectively to the surface of the metal thin film. In the case of His-tagging, the substrate is immobilized after treating with nickel chloride an array to which NTA has been introduced.

The present invention uses a chelate compound to monitor the phosphorylation of the substrate on the array specifically and sensitively. A chelate compound is generally a polydentate chelator complex or a chelating reagent complex which are coordinated to zinc, iron, cobalt, palladium, or a like metal ion. Preferable among them are compounds which selectively and reversibly bind to phosphoric acid. A polyamine-zinc complex is more preferable. A binuclear zinc complex containing a polyamine compound as a chelator is even more preferable. A hexamine binuclear zinc(II) complex containing a binuclear zinc(II) complex in its basic structure is yet more preferable.

A typical example of such a compound is a binuclear zinc complex of formula (I) containing as a chelator a polyamine compound containing 1,3-bis[bis(2-pyridylmethyl)amino]-2-hydroxy propanolate (IUPAC name: 1,3-bis[bis(2-pyridylmethyl)amino]-2-propanolatodizinc(II) complex) as a basic unit (note that the hydroxyl group in the propanol unit is a crosslinking chelator for two divalent zinc ions as an alcoholate. This example is however not limiting the invention in any particular manner.

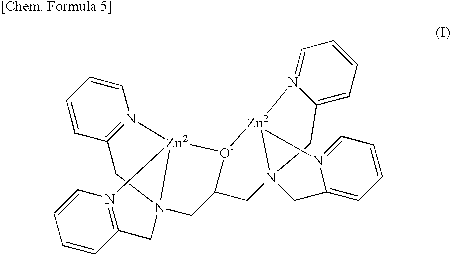

The complex used in the present invention may be synthesized from a commercially available compound by general chemical synthesis technology. As an example, the compound (Zn₂L) of formula (I) can be synthesized from commercially available 1,3-bis[bis(2-pyridylmethyl)amino]-2-hydroxypropane and zinc acetate as follows. A 10-M aqueous solution (0.44 mL) of sodium hydroxide is added to an ethanol solution (100 mL) containing 4.4 mmol of 1,3-bis[bis(2-pyridylmethyl)amino]-2-hydroxypropane. Zinc acetate dihydrate (9.7 mmol) is then added. The solvent is then removed in vacuo to obtain brown oil. Water (10 mL) is added to this residue to dissolve it. A 1-M aqueous solution of sodium perchlorate (3 equivalent amounts) is added dropwise while being heated to 70° C. The precipitating colorless crystal is filtered out and dried with heat to obtain at high yield the salt of diperchloric acid (Zn₂L—CH₃COO—.2ClO₄—.H₂O) of structural formula (I) to which an acetate ion is bound. The crystal contains one molecule of crystal water.

The ligand and receptor used in the present invention are preferably selected so that they can specifically recognize and bind to each other. Examples of such ligand/receptor combinations are biotin/avidin, biotin/streptavidin, steroid hormone/steroid hormone receptor, nucleic acid/transcription factor, and single-chain nucleic acid sequence/complementary single-chain nucleic acid sequence. The use of biotin as the ligand and avidin or streptavidin as the receptor is especially preferred among these combinations. The examples are however not limiting the invention.

The present invention is characterized by the use of the polyamine-zinc complex described above which is modified with a ligand (e.g., biotin). The ligand is not limited to biotin. It may be steroid hormone, nucleic acid, etc. The ligand is preferably modified with biotin with a straight-chain linker structure intervening between the ligand and the biotin. Its molecular weight is from 500 to 1,000, preferably from 600 to 900, more preferably from 700 to 900. A specific structural example is shown in formula (II). This is however not limiting the invention. It is not preferable in terms of stability and binding efficiency to the phosphoric acid if the biotin-modified polyamine-zinc complex has a molecular weight in excess of 1,000.

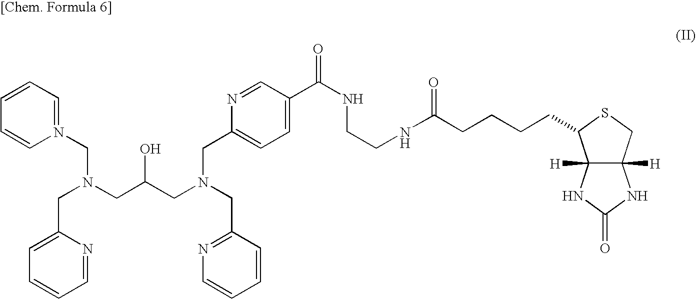

The concentration of the solution of the biotin-modified polyamine-zinc complex used the present invention is not limited in any particular manner and typically ranges from 1 μM to 10 M, preferably from 10 μM to 1 M, more preferably from 10 μM to 10 mM. The array may be treated with the solution in any manner. For example, a sufficient amount of the solution of the biotin-modified polyamine-zinc complex may be dropped so that the solution can spread across the array surface. Alternatively, the array may be immersed in the solution. A further alternative is to use a pump to deliver the solution so that it can contact the array surface. The array may be treated at room temperature or in incubation at 20 to 40° C. The treatment preferably lasts from about 10 minutes to 2 hours, preferably from 30 minutes to 1 hour.

In the present invention, the phosphorylation is generally detected by treating the array only with the biotin-modified polyamine-zinc complex. The detection sensitivity is however expected to increase if the treatment with the biotin-modified complex is followed by a treatment with a receptor, such as avidin or streptavidin. The receptor is not at all limited to avidin or streptavidin and may be a steroid hormone receptor, a transcription factor, a nucleic acid, etc. A treatment with streptavidin is preferred. The concentration of the avidin or streptavidin used in the treatment is not limited in any particular manner and typically ranges from 1 μM to 10 M, preferably from 10 μM to 1 M, more preferably from 10 μM to 10 mM. The array may be treated with the receptor in any manner similarly to the biotin-modified polyamine-zinc complex.

As a more preferred example, the detection sensitivity further increases if the treatment with avidin or streptavidin is accompanied by a treatment with an antibody which recognizes the avidin or streptavidin. The concentration of the antibody used in the treatment is not limited in any particular manner and ranges preferably from 0.01 to 10 μg/mL, more preferably from about 0.1 to μg/mL. The antibody may be either monoclonal or polyclonal. The monoclonal antibody is preferred in view of specificity. The array may be treated with the antibody in any manner similarly to the biotin-modified polyamine-zinc complex, avidin, and streptavidin.

The array may be treated sequentially, first with the biotin-modified polyamine-zinc complex, then followed by either avidin or streptavidin. Alternatively, the array may be treated directly with a complex of the biotin-modified polyamine-zinc complex and either avidin or streptavidin which is prepared in advance. In that case, the array may be further treated with an antibody which recognizes the avidin or streptavidin as in the foregoing case. The complex is preferably prepared by reacting the biotin-modified polyamine-zinc complex and either avidin or streptavidin at a mole ratio of 1:1 to 4:1. The product is preferably refined to remove unreacted substance; the product may however be applied as is.

These chelating compounds are advantageous because they can be synthesized by the method mentioned above at very low cost. They are also stable and easy to use because they can be stored at normal temperature and also advantageous in distribution. The compounds are far better than especially the detection methods involving an antibody in that the compounds are effective no matter which type of amino acid residue is to be phosphorylated and also that the reaction does not dependent on the amino acid sequence in the neighborhood of the phosphorylated amino acid.

The protein kinase may be one of various tyrosine kinases and serine/threonine kinases. The invention is applicable to any protein kinase. Basically, the invention is applicable to any protein kinase.

### EXAMPLES

The following will describe the present invention more specifically by means of examples. The examples however are not limiting the present invention in any particular manner.

### Example 1

(Immobilization of Peptide)

A four-arm PEG (SUNBRIGHT PTE-100SH made by NOF Corporation), having a thiol group as the terminus functional group, was dissolved in 7 mL of a mixed solution of ethanol and water (ratio=6:1, concentration=1 mM). The four-arm PEG molecule weighs 10,000 and has four PEG chains of substantially the same length extending from the center. The molecule exhibits very high hydrophilicity. All the four termini of the PEG are a thiol group. The molecule binds to metals, especially, gold. Chromium was vapor-deposited up to a 3 nm thickness on an SF15 glass slide (18 mm×18 mm and 2-mm thick). Gold was then vapor-deposited up to a 45 nm thickness. The gold-coated slide was immersed in the solution of the four-arm PEG thiol for 3 hours so that the four-arm PEG thiol could be immobilized on the whole gold basal plate.

The slide was covered with a photo mask and subjected to UV irradiation from a 500 W ultrahigh pressure mercury lamp (made by Ushio, Inc.) for 2 hours to remove the four-arm PEG thiol from UV-irradiated parts of the slide. The photo mask had 8×12=96 square holes measuring 500 μm on each side. The holes had a center-to-center pitch of 1 mm. The UV light passes through the holes of the photo mask and hits the slide, forming a pattern on the slide. The four-arm PEG remains in those parts which are not irradiated by the light. The parts will serve as a background section, or reference section, of the chip.

The slide was then immersed in a 1-mM ethanol solution of an 8-amino-1-octanethiol hydrochrolide (8-AOT made by Dojindo Laboratories) for 1 hour to form a self-assembled surface of 8-AOT in the UV-irradiated parts. A hetero difunctional polyethylene glycol (NHS-PEG-MAL made by Nektar) was dissolved in a phosphate buffer (20-mM phosphate, 150-mM NaCl, pH=7.2) to a concentration of 10 mg/mL. The NHS-PEG-MAL molecular weighs 3,400 and has a succinimide (NHS) group and a maleimide (MAL) group at its termini. The mixture was reacted with the 8-AOT on the surface of the gold for 2 hours. The amino groups of the 8-AOT react with the NHS groups of NHS-PEG-MAL. The MAL groups remain unreacted. Thus, the maleimide groups are introduced to the surface with the intervening PEG.

Five substrates for protein kinases (phosphorylated substrates and non-phosphorylated substrates) would be immobilized on the surface thus obtained. FIG. 1 shows at its bottom the amino acids sequences of the substrate peptides and a pattern of their immobilization. “Blank” indicates a blank spot where no peptide was immobilized. The substrate peptides were dissolved in respective phosphate buffers (20-mM phosphate, 150-mM NaCl, pH=7.2) to a rate of 1 mg/mL. The slide was then spotted with 10 nl of each solution using a MultiSPRinter™ spotter (made by Toyobo Co., Ltd.). Next, the slide was left at room temperature for 16 hours in a wet environment to let the immobilization reactions proceed. The maleimide groups formed on the chip surface and the thiol groups of the cysteine residues at the termini of the substrate peptides react, immobilizing the substrate peptides to the surface by covalent bonds.

(Blocking of Unreacted Maleimide Groups)

The surface on which the substrate peptides had been immobilized was washed in a phosphate buffer. Thereafter, to block unreacted maleimide groups, PEG thiol (SUNBRIGHT MESH-50H made by NOF Corporation) was dissolved in a phosphate buffer (20-mM phosphoric acid, 150-mM NaCl, pH=7.2) to a concentration of 1 mM, and 300 μL of the solution was dropped on the chip. Reactions were allowed to proceed at room temperature for 30 minutes. The PEG thiol used here had a molecular weight of 5,000.

(Detection of Phosphate Groups on Array)

After the blocking, the array was washed in PBS and water and treated with a biotin-modified polyamine-zinc complex. The biotin-modified polyamine-zinc complex used was Phos-tag™ BTL-104 (purchased from Nard Institute, Ltd.) of formula (II) below. Phos-tag™ BTL-104 was diluted to 25 μg/mL with a 10-mM HEPES-NaOH buffer (pH=7.4) containing 0.005% Tween 20, 10% (v/v) ethanol, 0.2 M sodium nitrate, and 1 mM zinc nitrate. The treatment was conducted at room temperature for 1 hour.

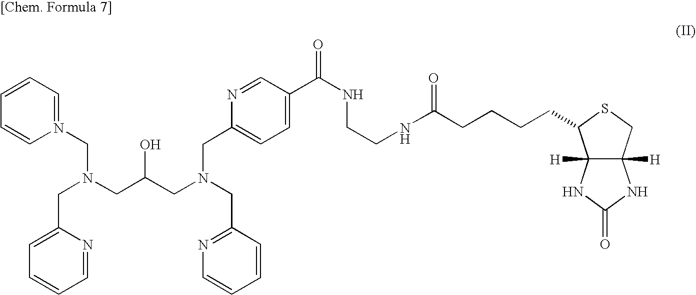

The treated array was washed in PBS and water and placed into an SPR instrument (MultiSPRinter™ made by Toyobo Co., Ltd.) for analysis. The same buffer as above, that is, the 10-mM HEPES-NaOH buffer (pH=7.4) containing 0.005% Tween 20, 10% (v/v) ethanol, 0.2 M sodium nitrate, and 1 mM zinc nitrate, was used as the running buffer. Streptavidin (made by MolecularProbes) was dissolved in the buffer to prepare 1, 5, 10, 50 μg/mL solutions. The array surface was treated with the solutions sequentially by continuously feeding them from a plunger pump (Model-021 made by Flom Co., Ltd.). The temperature was set to 30° C. The obtained sensorgram is shown at the top of FIG. 1. Significant increase in signal intensity is recognized for every phosphorylated substrate when compared to non-phosphorylated substrates although the intensity varies depending on the type of the phosphorylated substrate.

Results of SPR imaging are shown in the center of FIG. 1. For the SPR analysis, an image was taken on a CCD camera every 5 seconds. The images taken before and after reaction with streptavidin (hereinafter, may be referred to as “SA”) were subjected to subtractive processing using image processing software, Scion Image (Scion Corp.), to produce the illustrated results. Spots are found only at the sites where the phosphorylated substrates were immobilized. That confirms that Phos-tag™ BTL-104 was bound specifically.

### Example 2

The same operations were carried out as in example 1 up to the blocking of the unreacted maleimide group. The array was then washed in PBS and water and placed into an SPR instrument (MultiSPRinter™ made by Toyobo Co., Ltd.) for analysis. The same running buffer was used as in example 1. Phos-tag™ BTL-104 was dissolved in the running buffer to prepare 1, 5, 10 μg/mL solutions. The array surface was treated with the solutions sequentially by continuously feeding them from a plunger pump (Model-021 made by Flom Co., Ltd.). The temperature was set to 30° C. Thereafter, the array surface was washed in the running buffer being fed from the pump, and further treated with 1, 5, 10 μg/mL solutions of streptavidin in this order by feeding them from the pump. The obtained sensorgram is shown at the top of FIG. 2. In this case, significant increase in signal intensity is again recognized for all phosphorylated substrates when compared to non-phosphorylated substrates although the intensity varies depending on the type of the phosphorylated substrate. Results of SPR imaging are shown in the center of FIG. 2. The SPR imaging was carried out as in example 1. Similar trends are found from the results.

### Example 3

(Immobilization of Peptide)

The four-arm PEG thiol was bound to the gold basal plate as in example 1. Following that, the same patterning was carried out as in example 1, except that a different photo mask was used in the UV irradiation. The photo mask had 4×4=16 square holes measuring 500 μm on each side. The accompanying introduction of amino groups to the array surface and the formation of the maleimide group surface using a crosslinking agent were also carried out as in example 1. The slide was spotted manually with 0.1 μL of each substrate peptide. The substrates used were PKA (protein kinase A) substrate (the same as PKA(Ser) in FIG. 1), a positive control (pPKA) of which the serine residue had been phosphorylated, and a negative control (nPKA) of which the serine residue was replaced by an alanine residue. The substrates were immobilized as the pattern which is shown at the bottom of FIG. 3. Reactions following the spotting were carried out as in example 1.

(Blocking of Unreacted Maleimide Groups)

Blocking was carried out exactly as in example 1.

(Detection of Phosphate Groups on Array)

After the blocking, the array was washed in PBS and water as preparation for phosphorylation with PKA. 400 μL of a PKA solution was dropped on the array to allow reactions to proceed at 30° C. for 30 minutes. The PKA solution consisted of 1 μL of a PKA catalytic subunit (made by Promega), 375 μL of a 50-mM Tris-hydrochlorate buffer (pH=7.4), 20 μL of a 1-M solution of magnesium chloride, and 4 μL of a 10-mM ATP (made by Amersham Bioscience).

The array, having been reacted with PKA, is washed in PBS and water and treated with Phos-tag™ BTL-104 under the same conditions as in example 1. The array was then washed in PBS and water and placed into an SPR instrument (MultiSPRinter™ made by Toyobo Co., Ltd.) for analysis. The same running buffer was used as in example 1. The array was treated with 1, 5, 10 μg/mL solutions of streptavidin in this order by continuous feeding as in example 1. The obtained sensorgram and results of SPR imaging are shown in FIG. 3. The strongest binding signal is observed with the positive control. A substantially strong binding signal is observed with the PKA substrate too. Negligible signal changes are observed with the negative control and the blank. These results indicate that the phosphorylation of the PKA substrates on the array was successfully detected.

### Example 4

An array was fabricated in 96 spots format by immobilization in the same manner as in example 1, except that the array carried the same PKA substrate and positive and negative controls as those in example 3, as well as a cSrc substrate and its positive control. The substrate pattern is shown at the bottom of FIG. 4. Blocking, PKA reaction, and treatment with Phos-tag™ BTL-104 were carried out as in example 3. The array was washed in PBS and water and placed into an SPR instrument (MultiSPRinter™ made by Toyobo Co., Ltd.) for analysis. The same running buffer was used as in example 1. The array was treated with a solution of streptavidin (10 μg/mL) by feeding the solution. After confirming that signals made no more substantial increases, the array was washed in the running buffer being fed, and further treated with a 2.5-μg/mL anti-streptavidin antibody (made by Vector) by feeding. The obtained sensorgram and results of SPR imaging are shown in FIG. 4. A strong signal increase is observed with the positive controls for the PKA and cSrc substrates. A substantial signal increase is observed with the PKA substrate too. Almost no signal changes are observed with the negative control and the blank. Signal increases became more distinct as a result of the treatment with the anti-streptavidin antibody. The signal increases also indicate excellent specificity. These results confirm that the anti-streptavidin antibody has an excellent sensitizing effect.

### Example 5

An array on which a PKA substrate (threonine type), a PKC substrate (serine type), and a cSrc substrate (Yao; tyrosine type) shown in FIG. 1, both phosphorylated and non-phosphorylated, are immobilized was fabricated as in example 1. Thereafter, the same blocking was carried out as in example 1. The array then was placed into an SPR instrument (MultiSPRinter™ made by Toyobo Co., Ltd.). Meanwhile, a solution Phos-tag™ BTL-104 (50 μg/mL) and a solution of streptavidin (75 μg/mL) were mixed in equal amounts. Reactions were run at room temperature for 30 minutes to produce a complex. This complex solution was diluted 10 fold and 5 fold with the same running buffer as in example 1. The array was treated with the dilute solutions by continuous feeding. After confirming that signals made no more substantial increases, the array was washed in the running buffer being fed, and further treated with a 2.5-μg/mL anti-streptavidin antibody (made by Vector) by feeding. The obtained sensorgram is shown in FIG. 5. Specific signal increases are found only with the phosphorylated substrates.

### INDUSTRIAL APPLICABILITY

The method of the present invention enables very simple and quick analysis of kinetics of various protein kinases without a need for a special technique. If SPR is used together with the method, there is no need to use a label either, such as a fluorescent or radioactive substance. The use of a chelate compound reduces cost and enables easy handling. Moreover, owing to that use, the method is not affected by the type of phosphorylated amino acid or the amino acid sequence in its neighborhood. These are great advantages over conventional methods. Especially, the present invention enables comprehensive analysis of many types of protein kinase signals, which leads to effective profiling of intercellular protein kinase kinetics upon drug administration or upon the introduction of a gene of which the functions are unknown. Accordingly, the invention is expected to find applications in genome drug development, for example, to analyze functions of new genes and as drug research tools. The invention hence will make great contributions to industry.

1. A method of analysis of protein kinase activity, being characterized by, in judging phosphorylation using a peptide which is immobilized on a basal plate, contacting a chelate compound modified with a ligand with the object peptide on the basal plate.

2. The method of claim 1, wherein after the treating with the chelate compound modified with a ligand, the peptide is further treated with a receptor.

3. The method of claim 1, wherein after the treating with the receptor, the peptide is further treated with an antibody which recognizes the receptor.

4. A method of analysis of protein kinase activity, being characterized by, in judging phosphorylation using a peptide which is immobilized on a basal plate, forming a complex of a receptor and a chelate compound modified with a ligand and contacting the complex with the object peptide on the basal plate.

5. The method of claim 4, wherein after the treating with the complex, the peptide is further treated with an antibody which recognizes the receptor.

6. The method of claim 1, wherein the chelate compound modified with a ligand has a molecular weight of 500 to 1,000.

7. The method of claim 6, wherein the chelate compound modified with a ligand has a molecular weight of 600 to 900.

8. The method of claim 1, wherein: the ligand is biotin; and the receptor specific to that ligand is either avidin or streptavidin.

9. The method of claim 1, wherein the chelate compound is a polyamine-zinc complex.

10. The method of claim 1, wherein the chelate compound is a binuclear zinc complex containing a polyamine compound as a chelator.

11. The method of claim 1, wherein the chelate compound is a compound of formula (I):

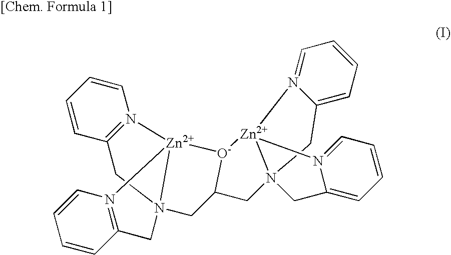

12. The method of claim 1, wherein the peptide is a substrate for at least one protein kinase selected from the group consisting of the cGMP-dependent protein kinase family, the cAMP-dependent protein kinase (PKA) family, the myosin light chain kinase family, the protein kinase C (PKC) family, the protein kinase D (PKD) family, the protein kinase B (PKB) family, the protein kinase family belonging to the MAP kinase (MAPK) cascade, the Src tyrosine kinase family, and the receptor tyrosine kinase family.

13. The method of claim 1, wherein an array is used which contains at least two peptides immobilized on a metal thin film, each peptide being a substrate for a different protein kinase.

14. The method of claim 1, wherein phosphorylation of at least one peptide which serves as a substrate for at least one protein kinase and is immobilized on a metal thin film in a basal plate of an array is detected by treating the at least one peptide with a nucleoside triphosphate and a test material which can contain a protein kinase.

15. The method of claim 1, wherein a phosphorylated peptide is detected by surface plasmon resonance (SPR).

16. The method of claim 1, wherein a phosphorylated peptide is detected by surface plasmon resonance imaging.

17. The method of claim 1, wherein the basal plate has a metal thin film, and the peptide is immobilized on the metal thin film.

18. The method of claim 1, wherein the peptide is immobilized on the basal plate, forming an array.

19. The method of claim 18, wherein two or more peptides are immobilized, forming an array.

20. The method of claim 18, wherein two or more peptides are immobilized, forming an array, the two or more peptides each containing an amino acid residue which is a combination of any two or more of serine, threonine, and tyrosine, the residue providing a site where the peptide is phosphorylated.

21. A kit for detecting phosphorylation using a peptide which is immobilized on a basal plate, the kit comprising: a chelate compound modified with a ligand; and a receptor specific to the ligand.

22. The kit of claim 21, the kit further comprising an antibody which recognizes the ligand.

23. The kit of claim 21, wherein: the ligand is biotin; and the receptor specific to that ligand is either avidin or streptavidin.

24. The kit of claim 21, wherein the chelate compound is a polyamine-zinc complex.

25. A kit for detecting phosphorylation using a peptide which is immobilized on a basal plate, the kit comprising a complex of a receptor and a chelate compound modified with a ligand.

26. The kit of claim 25, the kit further comprising an antibody which recognizes the ligand.

27. The kit of claim 25, wherein: the ligand is biotin; and the receptor specific to that ligand is either avidin or streptavidin.

28. The kit of claim 25, wherein the chelate compound is a polyamine-zinc complex.

29. The kit of claim 21, wherein the chelate compound is a binuclear zinc complex containing a polyamine compound as a chelator.

30. The kit of claim 21, wherein the chelate compound is a compound of formula (I):

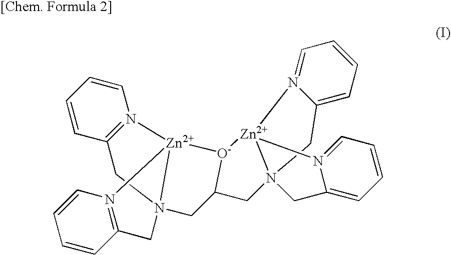

31. The kit of claim 21, wherein a phosphorylated form of the peptide is detected by surface plasmon resonance (SPR).

32. The kit of claim 21, wherein a phosphorylated form of the peptide is detected by SPR imaging.


SEQUENCE LISTING (6 sequences)

SEQ ID NO: 1 - protein, 10 aa, Artificial Sequence
  The sequence of designed peptide described in examples as substrate of PKA, Serine type
CGGLRRASLG

SEQ ID NO: 2 - protein, 10 aa, Artificial Sequence
  The sequence of designed peptide described in examples as substrate of PKA, Threonine type
CGGLRRATLG

SEQ ID NO: 3 - protein, 19 aa, Artificial Sequence
  The sequence of designed peptide described in examples as substrate of PKC
CGGAAKIQASFRGHMARKK

SEQ ID NO: 4 - protein, 10 aa, Artificial Sequence
  The sequence of designed peptide described in examples as substrate of cSrc kinase
CGIYGEFKKK

SEQ ID NO: 5 - protein, 10 aa, Artificial Sequence
  The sequence of designed peptide described in examples as substrate of cSrc kinase
CGGYIYGSFK

SEQ ID NO: 6 - protein, 10 aa, Artificial Sequence
  The sequence of designed peptide described in examples as negative control for substrate of PKA
CGGLRRAALG